(19) United States
(12) Patent Application Publication
(10) Pub. No.: US 2016/0033505 A1
(43) Pub. Date: Feb. 4, 2016

(54) Mobile Phase Test Strip Components, Conjugate Pad Pre-treatment Solutions, And Related Methods Thereof

(71) Applicant: Laborie Medical Technologies Canada ULC, Mississauga (CA)

(72) Inventor: Nevin Perera, Bedford (GB)

(21) Appl. No.: 14/448,646

(22) Filed: Jul. 31, 2014

(51) Int. Cl. G01N 33/573; G01N 33/553; G01N 33/543

(52) U.S. Cl. CPC G01N 33/573; G01N 33/54386; G01N 33/553; G01N 2333/96433

(57) ABSTRACT

A mobile phase test strip component is described herein including a conjugate pad. The conjugate pad includes a gold solution antibody having an antibody loading concentration of approximately 4 μg/OD and a gold OD of 5 to 6. The gold solution antibody is conjugated with a TRIS buffer solution and re-suspended with a dispensing buffer. The conjugate pad can include a high density polyester fiber pad material. A conjugate pad pre-treatment solution is also described, including at least one protein, at least one surfactant, at least one metal cation, and at least one polymer. A method for pre-treating a conjugate pad is also described.

### FIELD OF THE INVENTION

The invention generally relates to immunoassay test strip components, pre-treatment solutions, and related methods thereof. More specifically, the present invention relates to mobile phase test strip components, conjugate pad pre-treatment solutions, and related methods thereof with regard to prostate-related conditions in a human body.

### BACKGROUND OF THE INVENTION

Prostate-specific antigen (PSA) is a protein produced by cells of the prostate, a small gland located beneath the bladder in a male. Small amounts of PSA can generally be found circulating in the blood. However, for a variety of reasons, it can be important to regularly test and monitor PSA levels throughout an individual's life for increases, elevated levels, and/or further fluctuations.

Elevated levels of PSA in a man can be an indication of prostate cancer, an indication of an enlarged or inflamed prostate, and/or an indication of other potential non-cancerous related conditions. Generally speaking, the higher the PSA level, the greater the likelihood of a man developing prostate cancer. As a result, a PSA test has been widely used as one method of screening males for prostate cancer or prostate deficiencies.

The PSA test can be considered one of several immunoassay methods. Immunoassay can be defined as a procedure for detecting or measuring the presence of specific proteins or other substances through the use of an antibody or immunoglobulin. The substance detected by the immunoassay is often referred to as an “analyte” and is in many cases a protein. Immunoassay methods generally detect the presence of the analyte in a sample of blood or other bodily fluid.

In addition to cancer screening, the PSA test can also be used to monitor men undergoing treatment who have been diagnosed with prostate cancer or an enlarged prostate to determine, inter alia, whether the condition is responding to treatment and/or whether there is a change or recurrence with regard to said condition.

Nevertheless, regardless of the purpose or motivation of the PSA test, medical professionals need to have available to them PSA tests that can deliver consistently accurate measurements of PSA levels in order to provide the highest possible level of care to their patients.

In view of the foregoing, the present invention addresses these and other needs by providing mobile phase test strip components, conjugate pad pre-treatment solutions, and related methods thereof capable of providing accurate measurement of PSA for the detection and management of prostate-related conditions in a human body.

### SUMMARY OF THE INVENTION

According to at least one embodiment of the present invention, a mobile phase test strip component is described herein including a conjugate pad. The conjugate pad of at least one embodiment includes a gold solution antibody having an antibody loading concentration of approximately 4 μg/OD, where OD refers to optical density, and a gold OD in a range of 5 to 6. The gold solution antibody is conjugated with a TRIS buffer solution and re-suspended with a dispensing buffer. The conjugate pad of at least one embodiment further includes a high density polyester fiber pad material.

According to at least one embodiment of the present invention, a conjugate pad pre-treatment solution is described herein. The conjugate pad pre-treatment solution of at least one embodiment includes: at least one protein, at least one surfactant, at least one metal cation, and at least one polymer. The at least one protein is selected from the group consisting of Bovine serum albumin (BSA) and casein. The at least one surfactant is selected from the group consisting of polyethylene glycol dodecyl ether and polyethylene glycol sorbitan monolaurate. The at least one metal cation is selected from the group consisting of sodium tetraborate and potassium metaborate. The at least one polymer is selected from the group consisting of sucrose and polyvinyl alcohol.

According to at least one embodiment of the present invention, a method for pre-treating a conjugate pad is described herein. The method of at least one embodiment includes dissolving at least one solute in a pre-determined volume of solvent to form a pre-treatment solution. The pH of the pre-treatment solution is adjusted to a desired pH to form a pH-adjusted pre-treatment solution. The method of at least one embodiment further includes immersing a conjugate pad into the pH-adjusted pre-treatment solution. The immersed conjugate pad is then dried.

### BRIEF DESCRIPTION OF THE DRAWINGS

The present disclosure may be better understood, and its numerous features and advantages made apparent to those skilled in the art by referencing the accompanying drawing.

FIG. 1 is a flow diagram relating to a method of the present disclosure.

### DETAILED DESCRIPTION

As referenced above, elevated levels of PSA in a man can be an indication of prostate cancer, an indication of an enlarged or inflamed prostate, and/or an indication of other potential non-cancerous related conditions. Accordingly, for a variety of reasons, it can be useful and important to regularly test and monitor PSA levels of an individual throughout the individual's life for increases, elevated levels, and/or further fluctuations in PSA levels.

In addition to cancer screening, testing and monitoring PSA levels in an individual can also be used to monitor men undergoing treatment who have been diagnosed with prostate cancer or an enlarged prostate to determine, inter alia, whether the condition is responding to treatment and/or whether there is a change or recurrence with regard to said condition.

In view of the above, it would be beneficial for medical professionals to have the ability to conduct a PSA test that can deliver consistently accurate measurements of PSA levels in order to provide the highest possible level of care to all patients. In the context of cancer screening, it would be beneficial to have components and related methods available to conduct cancer screening tests that can greatly reduce and/or eliminate the occurrence of false-positive and false-negative cancer test results.

A false-positive cancer test result occurs when a individual's PSA level is elevated but no cancer is actually present. A false-positive test result may create unnecessary anxiety and distress for a man and his family and lead to additional medical procedures, such as a prostate biopsy, that can be harmful. As an example, possible negative side effects of biopsies include the possibility of serious infections, pain, and/or bleeding.

A false-negative cancer test result occurs when a man's PSA level is low even though he actually has prostate cancer. False-negative test results may give a man, his family, and his doctor a false assurance that he does not have cancer, when he may in fact have a cancer that requires immediate medical treatment.

Accordingly, different embodiments address these and other needs by providing mobile phase test strip components, conjugate pad pre-treatment formulations, and related methods thereof capable of providing accurate measurement of PSA for the detection and management of prostate-related conditions in a human body.

It was surprisingly found that at least one provides for, inter alia: (1) an overall improved stability of PSA tests when incorporated, for example, in a PSA test strip; (2) a gold conjugation process that is stable and robust; (3) an unexpectedly lessened gold OD (optical diameter); and (4) less variation in flow rates during testing when incorporating the invention in PSA testing protocols and/or PSA related test strips.

Immunoassay test strips can generally be divided into a solid phase and a mobile phase. Embodiments of the present invention include a mobile phase test strip component.

In mobile phase test strip component embodiments, the mobile phase test strip can include a conjugate pad. The conjugate pad can include a gold solution antibody. Suitable antibodies of the present invention include, for example, a PSA antibody.

Gold sol or gold colloid is ruby red in color and should generally remain this way when conjugated to an antibody. To conjugate, the antibody is first diluted using a buffer and/or a buffer solution, before being combined with the gold.

Suitable buffers and/or buffer solutions for conjugation of the gold solution antibody to gold colloid include a TRIS buffer solution. A TRIS buffer solution is a buffer made using the weak base tris(hydroxymethyl)aminomethane (HOCH₂)₃CNH₂, which is more commonly called “TRIS.” Accordingly, it will be appreciated that as used herein, “TRIS buffer solution” or “TRIS buffer,” and similar terms, are used to indicate a buffer or a buffer solution that is made using tris(hydroxymethyl)aminomethane (HOCH₂)₃CNH₂.

In mobile phase test strip component embodiments of the present invention, the buffer solution for conjugation of the antibody to gold colloid, such as the TRIS buffer solution, can have a pH of approximately 7.5 to 8.0, such as approximately 7.8, ±2.0%.

The buffer solution for conjugation of the antibody to gold colloid can also include TRIS buffer in a range of 2.5 to 100 mM or 0.0025 to 0.1 M, where M refers to moles of solute per liter of solution.

In cases where it is desired, the buffer diluted antibody solution can then be challenged with sodium chloride (e.g., a 1M sodium chloride solution) to determine how effective or how strong the binding is between the gold and the antibody. Weak conjugations where the buffer is at an incorrect pH will result in a color change of the gold sol from red to purple or black.

In mobile phase test strip component embodiments, the remaining gold-antibody complex can then be re-suspended with a dispensing buffer.

In embodiments, the dispensing buffer solution can include sucrose. Suitable amounts of sucrose include approximately 0.5 to 30.0%, based on a total weight of the dispensing buffer solution. In certain embodiments, the sucrose can be present in an amount of approximately 15%, based on a total weight of the dispensing buffer solution.

In terms of lower limits, the amount of sucrose present in the dispensing buffer solution can be at least approximately 0.5%, based on a total weight of the dispensing buffer solution, such as at least approximately 1.0%, at least approximately 2.0%, at least approximately 3.0%, at least approximately 3.5%, at least approximately 4.0%, at least approximately 5.0%, at least approximately 6.0%, at least approximately 7.0%, at least approximately 8.0%, at least approximately 9.0%, at least approximately 10.0%, at least approximately 10.5%, at least approximately 11.0%, at least approximately 12.0%, at least approximately 12.5%, at least approximately 13.0%, at least approximately 13.5%, at least approximately 14.0%, at least approximately 14.5%, and/or at least approximately 15.0%, based on a total weight of the dispensing buffer solution.

In terms of upper limits, the amount of sucrose present in the dispensing buffer solution can be no greater than approximately 30.0%, based on a total weight of the dispensing buffer solution, such as no greater than approximately 29.5%, no greater than approximately 29.0%, no greater than approximately 28.0%, no greater than approximately 27.0%, no greater than approximately 26.0%, no greater than approximately 25.5%, no greater than approximately 25.0%, no greater than approximately 24.0%, no greater than approximately 23.0%, no greater than approximately 22.0, no greater than approximately 21.0%, and/or no greater than approximately 20.0%, based on a total weight of the dispensing buffer solution.

The dispensing buffer solution in at least one embodiment can also include an amount of Bovine serum albumin (BSA). Suitable amounts of Bovine serum albumin (BSA) include approximately 0.25 to 20%, based on a total weight of the dispensing buffer solution, such as approximately 2.25% BSA, based on a total weight of the dispensing buffer solution.

In terms of lower limits, the amount of BSA present in the dispensing buffer solution can be at least approximately 0.25%, based on a total weight of the dispensing buffer solution, such as at least approximately 0.50%, at least approximately 0.75%, at least approximately 1.0%, at least approximately 1.25%, at least approximately 1.5%, at least approximately 1.75%, at least approximately 1.80%, at least approximately 1.90%, at least approximately 2.0%, at least approximately 2.15%, at least approximately 2.25%, at least approximately 3.0%, at least approximately 4.0%, and/or at least approximately 5.0% based on a total weight of the dispensing buffer solution.

In terms of upper limits, the amount of BSA present in the dispensing buffer solution can be no greater than approximately 20.0%, based on a total weight of the dispensing buffer solution, such as no greater than approximately 18.5%, no greater than approximately 17.0%, no greater than approximately 15.0%, no greater than approximately 14.0%, no greater than approximately 12.0%, no greater than approximately 10.5%, no greater than approximately 8.0%, no greater than approximately 7.0%, no greater than approximately 6.50%, no greater than approximately 5.0, no greater than approximately 4.0%, and/or no greater than approximately 3.0%, based on a total weight of the dispensing buffer solution.

The dispensing buffer solution can also include TRIS buffer in a range of 0.5 to 50 mM or 0.0005 to 0.05M, where M refers to moles of solute per liter of solution. In embodiments, the dispensing buffer can also have a pH of approximately 7.5 to 8.0, such as 7.8, ±2.0%

In mobile phase test strip component embodiments of the present invention, the gold-conjugated antibody can include an antibody and gold-conjugated in equal amounts, or equal volumes. In embodiments, the antibody can include a prostate specific antigen (PSA) antibody.

The gold-conjugated antibody of the present invention can have an antibody loading concentration of approximately 4 μg/OD, ±2%, where OD refers to optical density.

In mobile phase test strip component embodiments, the gold optical density can be in a range of 5 to 6, ±2%.

In terms of materials, conjugate pads in at least one embodiment can be made of a high density polyester fiber pad material.

In mobile phase test strip component embodiments of the present invention, the conjugate pad of the mobile phase test strip component can be hydrophilic in nature. However, it will be appreciated that in embodiments, the conjugate pad can be initially or naturally hydrophobic in nature. Therefore, in order to change the nature of the conjugate pad from an initial hydrophobic nature to that of hydrophilic, a treatment or a pre-treatment mechanism can be utilized.

Accordingly, in mobile phase test strip component embodiments, the conjugate pad is immersed in a pre-treatment solution and sprayed with a lectin solution, prior to adding the gold-conjugated antibody. The pre-treatment solution can serve the purpose of modifying or altering the naturally hydrophobic conjugate pad to be hydrophilic in nature.

In mobile phase test strip component embodiments, the pre-treatment solution can include: (1) at least one protein; (2) at least one surfactant; (3) at least one metal cation; and (4) at least one polymer. The at least one protein can function to stabilize lectin and conjugated colloidal gold, and assist in movement of rehydrated gold up an immunoassay strip utilizing the conjugate pad of the present invention. The at least one surfactant can function to alter the hydrophobic nature of the synthetic material. The at least one metal cation can function to regulate proper release of the detection conjugate. Lastly, the at least one polymer can function to stabilize the lectin and conjugated colloidal gold, as well as assist in movement of rehydrated fold up an immunoassay strip utilizing the conjugated pad of at least one embodiment.

The at least one protein of the pre-treatment solution can include Bovine serum albumin (BSA) and casein, and mixtures thereof.

The at least one surfactant of the pre-treatment solution can include polyethylene glycol dodecyl ether (also known as polyoxyethylene (23) lauryl ether), polyethylene glycol sorbitan monolaurate (also known as polyoxyethylenesorbitan monolaurate), and mixtures thereof.

Suitable polyethylene glycol dodecyl ether (also known as polyoxyethylene (23) lauryl ether) includes BRIJ®-35, manufactured by Sigma-Aldrich Corporation of St. Louis, Mo., USA. Suitable polyethylene glycol sorbitan monolaurate (also known as polyoxyethylenesorbitan monolaurate) include TWEEN® 20, manufactured by Sigma-Aldrich Corporation of St. Louis, Mo., USA.

The at least one metal cation of the pre-treatment solution can include sodium tetraborate, potassium metaborate, and mixtures thereof.

The at least one polymer of the pre-treatment solution can include sucrose, polyvinyl alcohol, and mixtures thereof.

In mobile phase test strip component embodiments of the present invention, the pre-treatment solution can also include a heterophilic blocker. The heterophilic blocker can be configured to actively, as opposed to passively, block heterophilic antibody interference in the mobile phase test strip component. For example, the heterophilic blocker can be configured to prevent interference from HAMA (Human Anti-Mouse Antibodies) and RF (Rheumatoid Factor), both of which can generate false positive test results.

Suitable heterophilic blockers of the present invention include TRU BLOCK® manufactured by MERIDIAN LIFE SCIENCE®, Inc. of Memphis, Tenn., USA.

Moreover, as referred to above, mobile phase test strip components in at least one embodiment can be incorporated into immunoassay test strips. Such immunoassay test strips can be utilized, for example, for the detection of PSA levels in an individual, cancer screening tests (e.g., prostate cancer), and for other medical and diagnostic tools and uses as well.

Embodiments also include, with reference to the above discussion of conjugate pad pre-treatment, a conjugate pad pre-treatment solution. The conjugate pad pre-treatment solution in at least one embodiment can include at least one protein, at least one surfactant, at least one metal cation, and at least one polymer.

Suitable proteins include Bovine serum albumin (BSA), casein, and mixtures thereof. Suitable surfactants include polyethylene glycol dodecyl ether (also known as polyoxyethylene (23) lauryl ether), polyethylene glycol sorbitan monolaurate (also known as polyoxyethylenesorbitan monolaurate), and mixtures thereof. Suitable metal cations include sodium tetraborate, potassium metaborate, and mixtures thereof. Suitable polymers include sucrose, polyvinyl alcohol, and mixtures thereof.

In terms of the selection of components of conjugate pad pre-treatment solution embodiments, the pre-treatment solution can include BSA, polyethylene glycol dodecyl ether surfactant, and sodium tetraborate metal cation.

In further embodiments, the conjugate pad pre-treatment solution can include 0.75% weight by volume percent polyethylene glycol dodecyl ether, 0.1% weight by volume percent BSA, and 5 mM sodium tetraborate metal cation.

Conjugate pad pre-treatment solutions in at least one embodiment can also include a 50 mM 4-(2-hydroxyethyl)-1-piperazineethanesulfonic acid. In certain embodiments, the 50 mM 4-(2-hydroxyethyl)-1-piperazineethanesulfonic acid has a pH of about 7 to 8.

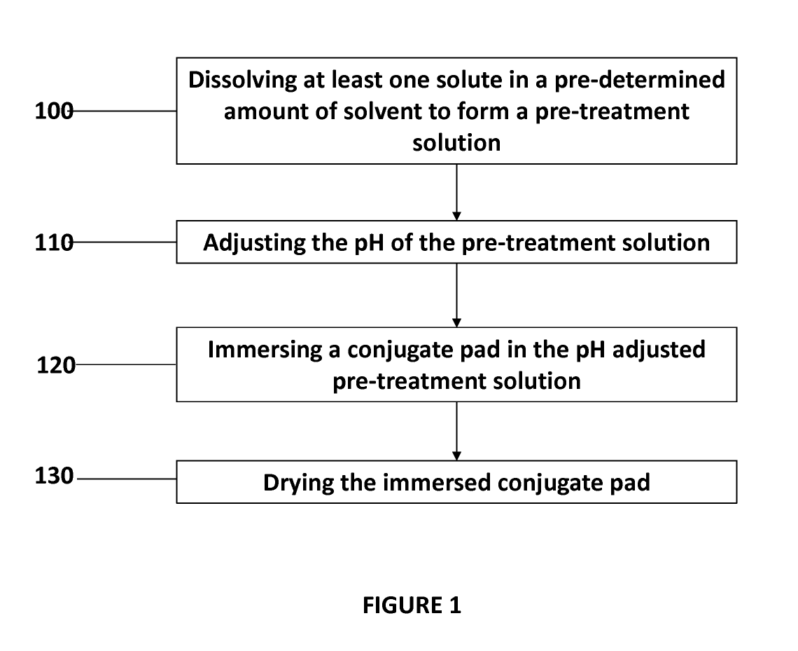

According to at least one embodiment, a method for pre-treating a conjugate pad is provided. With reference to FIG. 1 methods for pre-treating a conjugate pad can include dissolving at least one solute in a pre-determined volume of solvent to form a pre-treatment solution 100.

Suitable pre-treatment solutions for methods include those described above. Therefore, in the interests of succinctness and improved readability, the above-description with regard to pre-treatment solutions apply equally to methods of the present invention, is incorporated herein by reference in its entirety, and will not be repeated herein.

Methods of pre-treating a conjugate pad according to at least one embodiment include adjusting a pH of the pre-treatment solution 110 to a desired pH to form a pH-adjusted pre-treatment solution. The conjugate pad is then immersed into the pH-adjusted pre-treatment solution 120. In certain method embodiments, the conjugate pad is immersed in the pre-treatment solution for at least four minutes, such as at least five minutes, at least 6 minutes, at least 7 minutes, at least 8 minutes, and/or at least nine minutes.

The immersed conjugate pad 130 is also dried. The drying of the immersed conjugate pad 130 may be done via application or exposure to a heat source or an elevated temperature. Alternatively, the drying 130 can be done at approximately room temperature. In certain method embodiments, the drying can be performed for at least one hour, and at a temperature of approximately 45° to 50° C.

In other alternative embodiments, the drying 130 can also be performed by passing the immersed conjugate pad through at least one drying tower. The at least one drying tower (e.g., two drying towers) can be set to pre-determined temperatures. In cases where it is desired, the dried conjugate pad (e.g., conjugate pad that has been passed through at least one dryer) can be placed in an incubator to further ensure dryness of the conjugate pad. In embodiments, the conjugate pad can be placed in the incubator for a pre-determined period of time, at a pre-determined temperature.

In a further embodiment, the conjugate pad is sprayed with a double lectin spray.

Moreover, it will be appreciated that in methods of the present invention, the conjugate pad that is being immersed is initially hydrophobic in nature. After the pre-treating is completed, the resulting pre-treated conjugate pad is hydrophilic in nature.

As used herein “substantially”, “relatively”, “generally”, “about”, and “approximately” are relative modifiers intended to indicate permissible variation from the characteristic so modified. They are not intended to be limited to the absolute value or characteristic which it modifies but rather approaching or approximating such a physical or functional characteristic.

In this detailed description, references to “one embodiment”, “an embodiment”, or “in embodiments” mean that the feature being referred to is included in at least one embodiment of the invention. Moreover, separate references to “one embodiment”, “an embodiment”, or “embodiments” do not necessarily refer to the same embodiment; however, neither are such embodiments mutually exclusive, unless so stated, and except as will be readily apparent to those skilled in the art. Thus, the invention can include any variety of combinations and/or integrations of the embodiments described herein.

The terminology used herein is for the purpose of describing particular embodiments only and is not intended to be limiting of the invention. As used herein, the singular forms, “a”, “an” and “the” are intended to include the plural forms as well, unless the context clearly indicates otherwise. It will be further understood that the root terms “include” and/or “have”, when used in this specification, specify the presence of stated features, integers, steps, operations, elements, and/or components, but do not preclude the presence or addition of at least one other feature, integer, step, operation, element, component, and/or groups thereof.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus.

As used herein, and unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

The corresponding structures, materials, acts, and equivalents of all means plus function elements in the claims below are intended to include any structure, or material, for performing the function in combination with other claimed elements as specifically claimed. The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments.

The terminology used herein was chosen to best explain the principles of the embodiments, the practical application or technical improvement over technologies found in the marketplace, or to enable others of ordinary skill in the art to understand the embodiments disclosed herein.

It will be appreciated that not all of the features, components and/or activities described above in the general detailed description in relation to embodiments of the present disclosure or the examples are required, that a portion of a specific feature, component and/or activity may not be required, and that one or more further features, components and/or activities may be required, added or performed in addition to those described. Still further, the orders in which activities are listed are not necessarily the order in which they are performed.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination.

In the foregoing, reference to specific embodiments and the connections of certain components is illustrative. It will be appreciated that reference to components as being coupled or connected is intended to disclose either direct connection between said components or indirect connection through one or more intervening components as will be appreciated to carry out the methods as discussed herein. As such, the above-disclosed subject matter is to be considered illustrative, and not restrictive, and the appended claims are intended to cover all such modifications, enhancements, and other embodiments, which fall within the true scope of the present invention.

Further, references to values stated in ranges include each and every value within that range, and the endpoints of said ranges. Thus, to the maximum extent allowed by law, the scope of the present invention is to be determined by the broadest permissible interpretation of the following claims and their equivalents, and shall not be restricted or limited by the foregoing detailed description.

What is claimed is:

1. A mobile phase test strip component comprising:
a conjugate pad, the conjugate pad including:
a gold solution antibody having an antibody loading concentration of approximately 4 μg/OD, where OD refers to optical density; and
a gold OD in a range of 5 to 6, wherein the gold solution antibody is conjugated with a TRIS buffer solution and re-suspended with a dispensing buffer, and
wherein the conjugate pad comprises a high density polyester fiber pad material.

2. The mobile phase test strip component of claim 1, wherein the gold solution antibody includes an antibody and gold, conjugated in equal volumes.

3. The mobile phase test strip component of claim 1, wherein the dispensing buffer includes approximately 0.5 to 30.0% sucrose, based on a total weight of the dispensing buffer solution.

4. The mobile phase test strip component of claim 1, wherein the dispensing buffer solution includes approximately 0.25% to 20.0% Bovine serum albumin (BSA), based on a total weight of the dispensing buffer solution.

5. The mobile phase test strip component of claim 1, wherein the dispensing buffer includes 0.5 to 50 mM or 0.0005 to 0.05M TRIS buffer.

6. The mobile phase test strip component of claim 1, wherein the conjugate pad is hydrophilic in nature.

7. The mobile phase test strip component of claim 1, wherein the conjugate pad is immersed in a pre-treatment solution prior to adding the gold-conjugated antibody.

8. The mobile phase test strip component of claim 7, wherein the pre-treatment solution includes:
at least one protein selected from the group consisting of Bovine serum albumin (BSA), casein, and mixtures thereof;
at least one surfactant selected from the group consisting of polyethylene glycol dodecyl ether, polyethylene glycol sorbitan monolaurate, and mixtures thereof;
at least one metal cation selected from the group consisting of sodium tetraborate, potassium metaborate, and mixtures thereof; and
at least one polymer selected from the group consisting sucrose, polyvinyl alcohol, and mixtures thereof.

9. The mobile phase test strip component of claim 7, wherein the pre-treatment solution includes a heterophilic blocker configured to actively block heterophilic antibody interference in the mobile phase test strip component.

10. The mobile phase test strip component of claim 1, wherein the antibody includes a prostate specific antigen (PSA) antibody.

11. An immunoassay test strip comprising the mobile phase test strip component of claim 1.

12. A conjugate pad pre-treatment solution comprising:
at least one protein selected from the group consisting of Bovine serum albumin (BSA), casein, and mixtures thereof;
at least one surfactant selected from the group consisting of polyethylene glycol dodecyl ether, polyethylene glycol sorbitan monolaurate, and mixtures thereof;
at least one metal cation selected from the group consisting of sodium tetraborate, potassium metaborate, and mixtures thereof; and
at least one polymer selected from the group consisting sucrose, polyvinyl alcohol, and mixtures thereof.

13. The conjugate pad pre-treatment solution of claim 12, wherein the at least one protein is BSA, the at least one surfactant is polyethylene glycol dodecyl ether, and the at least one metal cation is sodium tetraborate.

14. The conjugate pad pre-treatment solution of claim 12, further comprising a 50 mM 4-(2-hydroxyethyl)-1-piperazineethanesulfonic acid.

15. The conjugate pad pre-treatment solution of claim 14, wherein the 50 mM 4-(2-hydroxyethyl)-1-piperazineethanesulfonic acid includes a pH of approximately 7 to 8.

16. The conjugate pad pre-treatment solution of claim 12, further comprising 0.75% weight by volume percent polyethylene glycol dodecyl ether, 0.1% weight by volume percent BSA, and 5 mM sodium tetraborate.

17. A method for pre-treating a conjugate pad comprising:
dissolving at least one solute in a pre-determined volume of solvent to form a pre-treatment solution;
adjusting a pH of the pre-treatment solution to a desired pH to form a pH-adjusted pre-treatment solution;
immersing a conjugate pad into the pH-adjusted pre-treatment solution; and
drying the immersed conjugate pad.

18. The method of claim 17, wherein the drying is performed for at least one hour at approximately 45 to 50° C.

19. The method of claim 17, wherein the pre-treatment solution includes at least one protein selected from the group consisting of Bovine serum albumin (BSA) and casein;
at least one surfactant selected from the group consisting of polyethylene glycol dodecyl ether and polyethylene glycol sorbitan monolaurate;
at least one metal cation selected from the group consisting of sodium tetraborate and potassium metaborate; and
at least one polymer selected from the group consisting sucrose and polyvinyl alcohol.

20. The method of claim 17, wherein the conjugate pad being immersed is hydrophobic, and the resulting pre-treated conjugate pad is hydrophilic.
